(19) United States
(12) Patent Application Publication
(10) Pub. No.: US 2017/0151385 A1
(43) Pub. Date: Jun. 1, 2017

(54) DRIP CHAMBER FOR ADMINISTERING A MEDICAL FLUID

(71) Applicant: Fresenius Kabi Deutschland GMBH, Bad Homburg (DE)

(72) Inventor: Harry Pütter, Bad Salzschirf (DE)

(21) Appl. No.: 15/310,365

(22) PCT Filed: Apr. 20, 2015
(86) PCT No.: PCT/EP2015/058496

§ 371 Date: Nov. 10, 2016

(30) Foreign Application Priority Data
May 28, 2014 (EP) 14170171.4

(51) Int. Cl. A61M 5/14; B29C 45/00; B29C 45/16; A61M 5/165

(52) U.S. Cl. CPC A61M 5/1411; A61M 5/165; B29C 45/0001; B29C 45/1676; A61M 2005/1657; A61M 2207/00; B29L 2031/753

(57) ABSTRACT

A drip chamber (1) for administering a medical fluid, comprises a chamber element (30) forming a chamber (300), an inlet (20) for letting a medical fluid into the chamber (300), and a connector (31) connected to the chamber element (30) at an outlet of the drip chamber (1). A tube (4) is connectable to the connector (31) for letting the medical fluid out of the chamber (300). Herein, the connector (31) is formed in one piece on the chamber element (30) by using injection molding, wherein the chamber element (30) is formed from a first material and the connector (31) is formed from a second material which is different from the first material. In this way a drip chamber is provided which allows for an easy, cost-efficient production and at the same time is easily squeezable and may provide a reliable, durable connection to a tube.

The invention relates to a drip chamber for administering a medical fluid according to the preamble of claim 1 and to a method for producing a drip chamber for administering a medical fluid.

A drip chamber of this kind comprises a chamber element forming a chamber therein. The drip chamber further comprises an inlet for letting a medical fluid into the chamber and an outlet for letting the medical fluid out of the chamber. A connector is connected to the chamber element at the outlet, a tube being connectable to the connector. Via the tube, the medical fluid may be guided for example to a patient.

A drip chamber may, for example, have the purpose to allow gas (such as air) to rise out from a fluid so that it is not passed down-streams. The use of a drip chamber furthermore allows to estimate the rate at which a fluid is administered. In particular, for a fluid of given viscosity, drips from a hole of known size will have an identical volume, such that the number of drips in a minute for example can be counted to obtain an estimate of the flow rate. Within an infusion set, the flow rate herein may for example be controlled by a clamp on a tube, effecting the flow resistance of the tube.

A drip chamber of this kind may be used for example for the infusion of medical fluids such as drugs or another liquid intravenously into a patient and for this purpose may be part of an infusion set. A drip chamber of this kind may also be used, for example, for the transfusion or the parenteral nutrition.

Within a drip chamber of this kind, it generally is desired to provide a squeezable chamber element which can be squeezed with low force such that a user, for example a nurse, may fill the drip chamber by squeezing the chamber element. As a further requirement, the chamber element should be transparent such that a user has a clear view into the chamber and can make out whether a medical fluid has filled the chamber or not.

The connector of the drip chamber, on the other hand, must be constituted such that it can provide a secure, durable connection to a tube, for example a PVC tube. For this purpose, the connector must exhibit a sufficient stiffness such that a tube can for example be glued to the connector, without the gluing connection being affected by for example PVC migration effects.

In a drip chamber known from U.S. Pat. No. 3,003,500, a chamber element is produced from a first, elastic material. A connector is connected to the chamber element at an outlet site and is constituted to provide a connection to an adjoining tube.

A drip chamber described in GB 717,733 comprises a tubular barrel of transparent, flexible, resilient plastic. The barrel is sufficiently rigid to retain its cylindrical shape under its own weight, but only relatively slight forces are necessary to collapse it. The lower end of the barrel is closed by a plastic reducing cap providing a connection to a tube.

U.S. Pat. No. 4,136,692 describes a drip chamber in which a bottom cap is connected to a chamber element providing a connection to a tube.

From U.S. Pat. No. 5,489,385, a drip chamber is known which comprises a chamber element fabricated by injection molding. Integrally formed from the same material with chamber element is a connector for connecting a tube to the drip chamber.

It is an object of the instant invention to provide a drip chamber which allows for an easy, cost-efficient production and at the same time is easily squeezable and may provide a reliable, durable connection to a tube.

This object is achieved with a drip chamber comprising the features of claim 1.

Accordingly, the connector of the drip chamber is formed in one piece on the chamber element by using injection molding, wherein the chamber element is formed from a first material and the connector is formed from a second material which is different from the first material.

This is based on the idea to form the connector and the chamber element of the drip chamber in one piece, but nevertheless of two materials. For this, the connector is formed on a chamber element using an injection molding technique.

Injection molding is understood as a manufacturing process for producing parts by injecting material into a mold. Material herein is fed into a mold cavity of the mold, where it cools and hardens to the configuration of the cavity.

For the instant drip chamber, the connector is formed by injection molding preferably in a separate molding step. Hence, in a first molding step the chamber element is formed, and in a subsequent, separate molding step the connector is molded from a different material on the chamber element. The connector, hence, is formed using a two-component injection molding technique using (at least) two different materials and providing as a result an integral piece forming the chamber element and the connector.

By employing a two-component injection molding technique, the connector and the chamber element are formed from different materials. Herein, preferably the first material forming the chamber element has a smaller modulus of elasticity than the second material forming the connector.

The material of the chamber element, hence, is less stiff as compared to the material of the connector. The chamber element thus may have a reduced stiffness as compared to the connector. The chamber element, in particular, may have a sufficient elasticity and flexibility such that it may, in a resilient manner, be squeezed to perform a pumping action on the drip chamber in order to cause a flow into the drip chamber.

The connector, in contrast, may be formed such that it has an increased stiffness as compared to the chamber element, such that a sufficiently stiff element is provided for connecting a tube to the connector in a reliable, durable manner.

By using a two-component injection molding technique, the production of a drip chamber may become easy and cost-efficient. In addition, it also becomes possible to form, in particular, the chamber element with a uniformly thin wall thickness, which may be thinner than for conventional drip chambers.

This is due to the fact that the connector may be integrally formed in one piece on the chamber element in a separate molding step. In conventional production methods forming a chamber element of a drip chamber by injection molding, as it is done for example in U.S. Pat. No. 5,489,385, a mold core of a molding tool is connected, in the region of the outlet, only via a thin pin to the opposite mold side. Such pin forms the outlet opening and correspondingly has a diameter equal to the diameter of the outlet opening. Because during injection a molding material is injected with high pressure, the pin may be deformed, possibly leading to a non-uniform wall thickness of the chamber element. This may result in a non-uniform flexibility and elasticity which may haptically be felt by a user when squeezing the drip chamber.

In contrast, because the connector, according to the instant invention, may be formed in a separate molding step using a two-component injection molding technique, the chamber element may be formed in a first molding step having a uniform, thin wall thickness. In particular, no thin pin for connecting a mold core to a surrounding molding tool is required. The connector may then be produced in an easy, reliable manner in the second molding step on the previously formed chamber element.

The first material (of the chamber element) and the second material (of the connector) may, for example, be thermoplastic polymers having different moduli of elasticity.

The first material may, for example, be a polystyrene or styrene butadiene block polymer of a first type, whereas the second material may be a polystyrene of a second type having a higher modulus of elasticity as compared to the polystyrene of the first type. The polystyrene of the first type, hence, is softer as compared to the stiffer polystyrene material of the second type.

Alternatively, the first material may be a polystyrene or styrene butadiene block polymer and the second material may be a methyl methacrylate acrylonitrile butadiene styrene (MABS), wherein again the first material has preferably a smaller modulus of elasticity than the second material.

The connector, in one embodiment, is arranged at a side of a chamber element opposite the inlet. The connector is placed at the outlet of the drip chamber and provides a connection to a tube which can be connected to the connector, for example, by gluing.

For receiving the tube, the connector may, for example, comprise a reception opening into which an end of the tube can be inserted. The reception opening, beneficially, adjoins an outlet opening formed in the connector or in the chamber element for providing a flow connection to the chamber such that a medical fluid may flow from the chamber through the outlet opening into a tube connected to the connector.

Beneficially herein, the reception opening has a larger diameter as compared to the diameter of the outlet opening. Preferably, the diameter of the reception opening corresponds to the outer diameter of a tube to be inserted in the reception opening, such that the tube can be received in the reception opening in an accurately fitting manner. The diameter of the outer opening, in contrast, preferably matches the inner diameter of the lumen of the tube, such that no step is formed between the outlet opening and the inner lumen of the tube when the tube is connected to the connector. The outlet opening and the inner lumen of the tube, hence, have the same diameter and align with each other, which has the advantage that the risk for the generation of bubbles when a fluid flows into the tube is reduced. Further, the maximum flow rate out of the drip chamber may be improved.

In one embodiment, the connector comprises a stub section forming the reception opening. The stub section herein may, for example, be connected to a bottom section of the chamber element via a cone section, the cone section tapering from the bottom section towards the stub section such that a medical fluid is guided towards the stub section and hence towards a tube connected to the connector.

The connector is formed on the chamber element using injection molding. Herein, the integral connection between the connector and the chamber element may, for example, be formed via a connecting section of the connector which circumferentially surrounds the chamber element at its bottom section. The chamber element, for example, may have a generally cylindrical shape (in a state in which it is not deformed by squeezing). The bottom section may correspond a circumferential edge region at a lower end of the chamber element, the bottom section being circumferentially surrounded by the connection section which, hence, at least partially encompasses the bottom section.

In another embodiment, the chamber element may comprise a stub section which extends into the connector and forms an insertion opening for receiving an end of the tube. The stub section of the chamber element may be overmolded by the connector, such that the stub section of the connector may surround the stub section of the chamber element. The stub section of the connector herein may extend axially longer (protruding from a bottom of the chamber element), wherein the tube may be received both in the reception opening of the stub section of the connector and the insertion opening of the stub section of the chamber element.

Beneficially, the insertion opening adjoins an outlet opening formed in the stub section of the chamber element for providing a flow connection from the chamber towards the tube. In order to avoid turbulences of a flow from the chamber into the tube, the insertion opening herein has a larger diameter, preferably corresponding to the outer diameter of the tube, as compared to the diameter of the outlet opening, which preferably matches the diameter of the inner lumen of the tube.

The drip chamber serves for example to allow gas to rise out from a fluid so that it is not passed down-streams. In addition, a filtering membrane may be provided in the drip chamber, such that furthermore a filtering of a medical fluid to be passed through the drip chamber is achieved. The membrane may be arranged, with a circumferential membrane edge, in-between a face of the chamber element and a face of the connector. The filter membrane serves to separate the chamber from the outlet of the drip chamber such that a fluid must pass through the membrane when flowing out of the drip chamber. By placing the circumferential membrane edge between a circumferential face, for example an end face of the chamber element, and a circumferential face of the connector, the membrane may be integrally fastened within the drip chamber when molding the connector on the chamber element. No separate step of fixing the membrane in the drip chamber is necessary. In particular, no gluing or welding of the membrane is required.

Alternatively, the membrane may also be connected to a circumferential face of the chamber element or the connector for example by gluing or welding it to the associated face. The membrane, hence, is fixed in a separate step, for example by hot stamping it onto the associated face of the chamber element or the connector.

The object is also achieved by a method for producing a drip chamber for administering a medical fluid, the drip chamber comprising:
a chamber element forming a chamber,
an inlet for letting a medical fluid into the chamber, and
a connector connected to the chamber element at an outlet, wherein a tube is connectable to the connector for letting the medical fluid out of the chamber.

Herein, the connector is formed in one piece with the chamber element using a two-component injection molding technique, wherein the chamber element is formed from a first material and the connector is formed from a second material which is different from the first material.

The advantages and advantageous embodiments described above for the drip chamber equally apply also to the method for producing the drip chamber. It hence shall be referred to the above.

In particular, the connector may be formed by injection molding in a separate step on the chamber element such that the chamber element and the connector are formed in separate injection molding steps. For the separate molding steps, herein different molding tools forming different molding cavities may be used. This allows for producing the chamber element with a uniformly thin wall thickness in a first step and a sufficiently rigid connector on the preferably elastic, flexible chamber element in a second step.

The idea underlying the invention shall subsequently being described in more detail with regard to the embodiments shown in the figures. Herein:

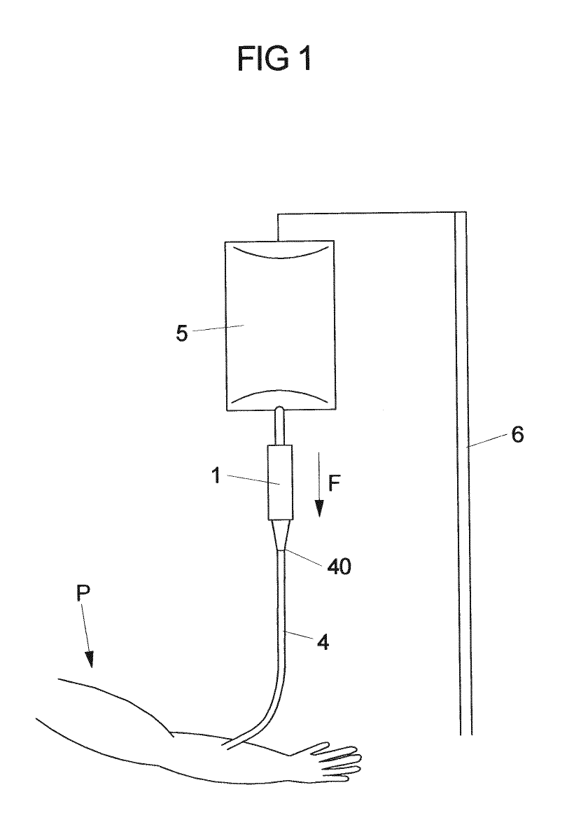

FIG. 1 shows a schematic view of an infusion set for intravenously infusing a medical fluid to a patient, the infusion set employing a drip chamber;

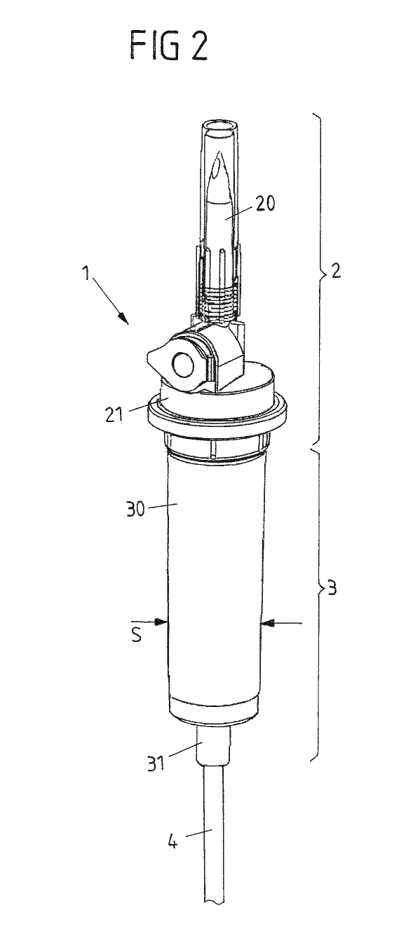

FIG. 2 shows a perspective view of a drip chamber;

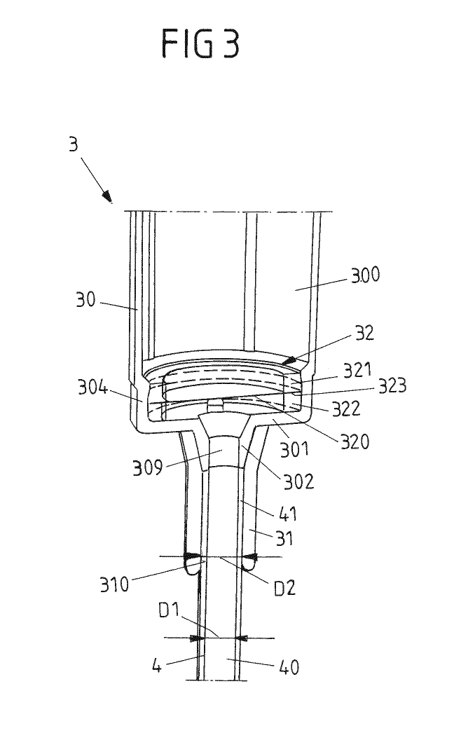

FIG. 3 shows a perspective, cut view of a first embodiment of a drip chamber according to the invention;

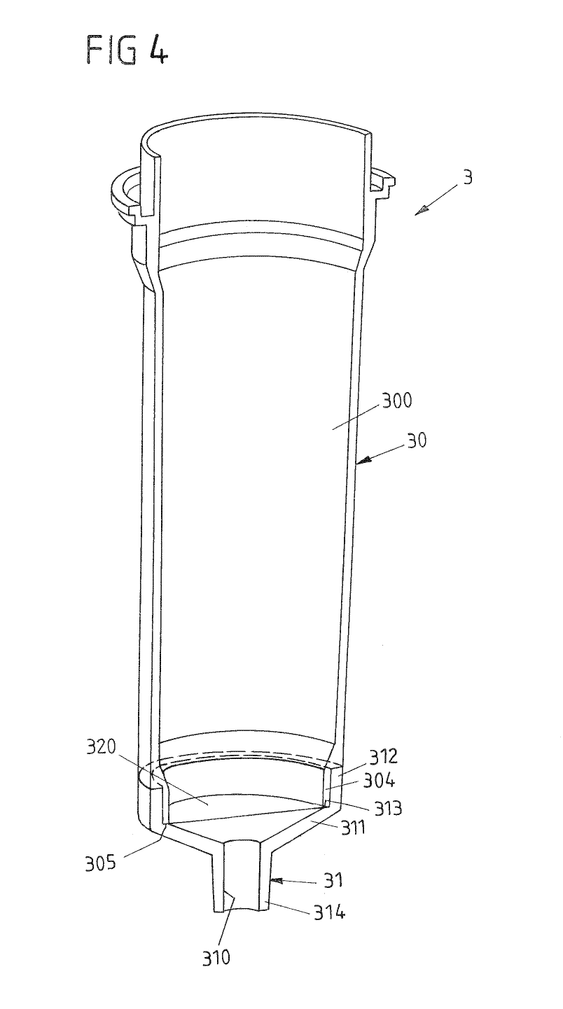

FIG. 4 shows a perspective, cut view of a second embodiment of a drip chamber;

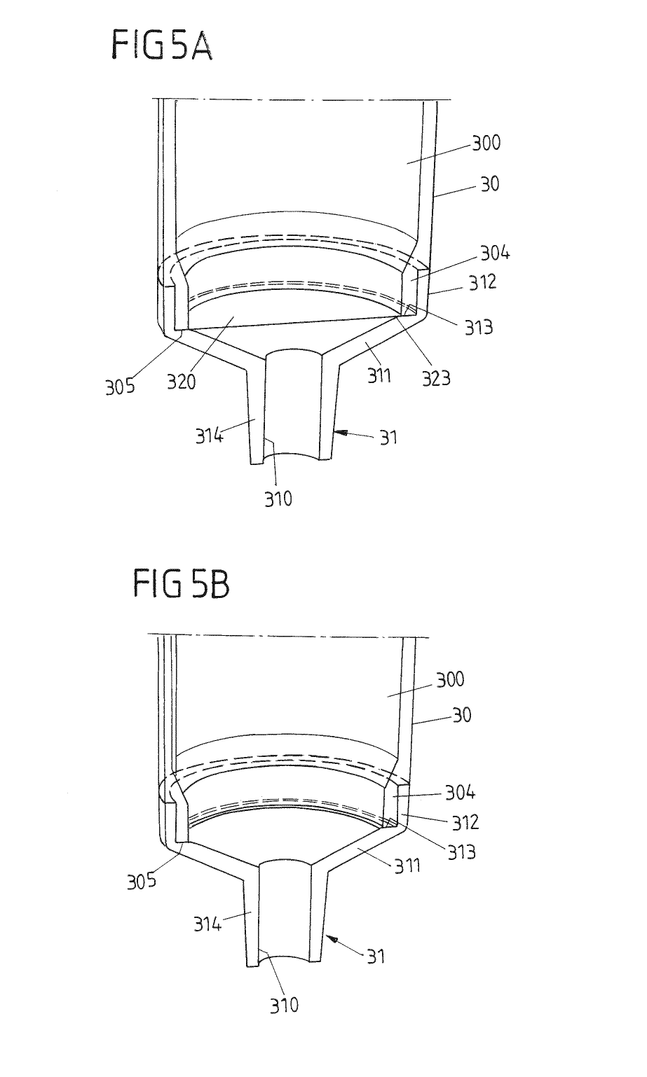

FIG. 5A shows a lower portion of the view of FIG. 4 in an enlarged way;

FIG. 5B shows the view of FIG. 5A without a membrane arranged in-between a chamber element and a connector;

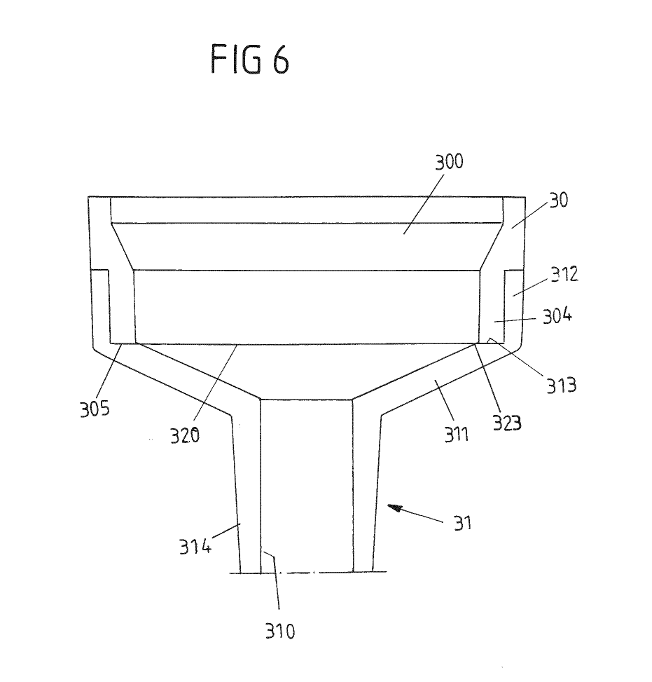

FIG. 6 shows a planar, cut view of the view of FIG. 5A;

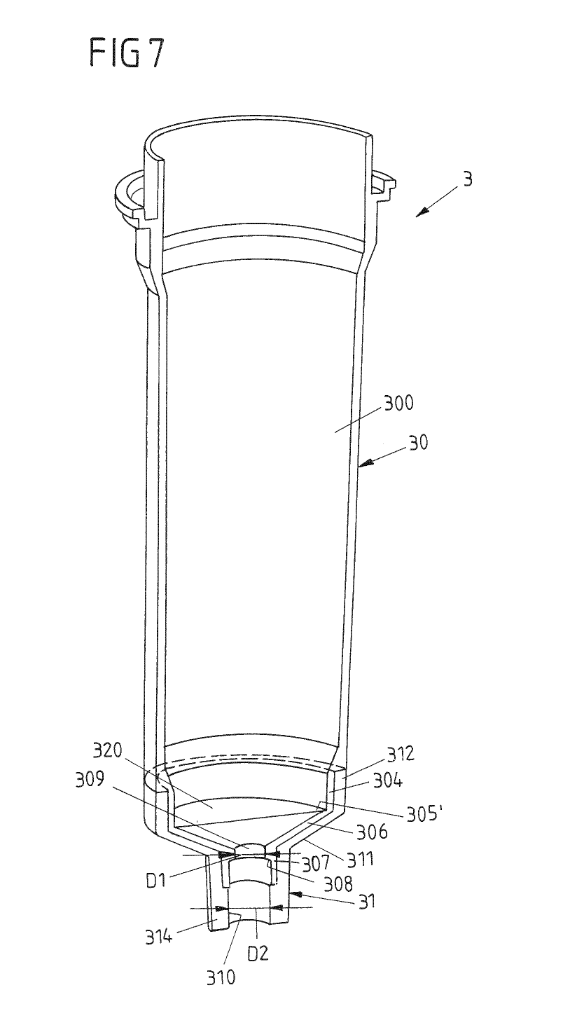

FIG. 7 shows a perspective, cut view of a third embodiment of a drip chamber;

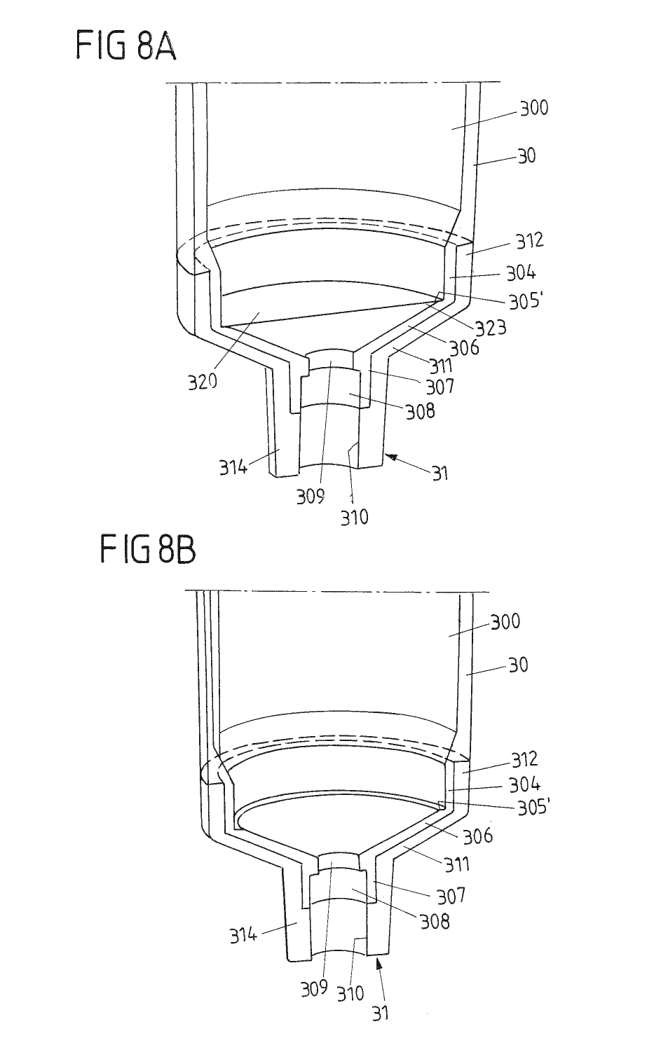

FIG. 8A shows a bottom portion of the view of FIG. 7;

FIG. 8B shows the view of FIG. 8A without a membrane; and

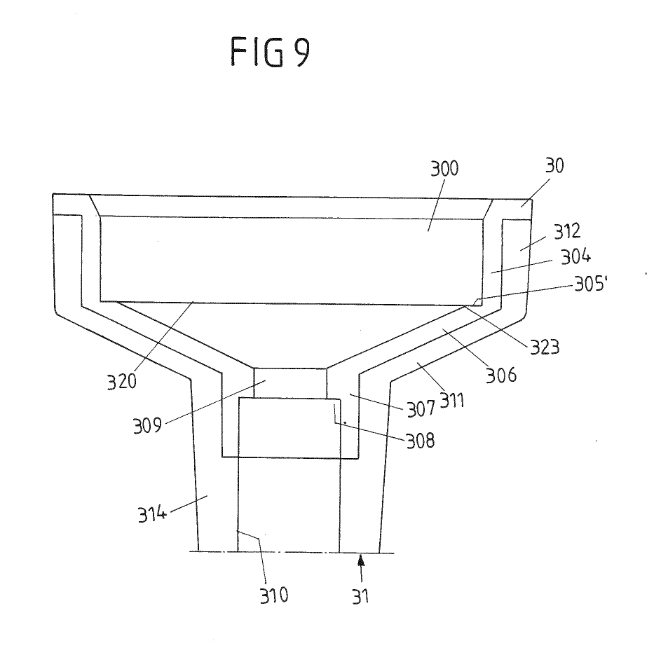

FIG. 9 shows a planar, cut view of the bottom portion of the drip chamber.

FIG. 1 shows a schematic view of an infusion set for administering a medical fluid out of a container 5, for example in the shape of a flexible bag, to a patient P. The infusion set comprises a drip chamber 1 and a tube 4 connected to the drip chamber 1 for administering the medical fluid out of the container 5 in a flow direction F to the patient P. The container 5 may for example be arranged on a stand 6 at a bed side of the patient P in a hospital environment.

The drip chamber 1 serves in particular to allow gas, for example air, to rise out of the medical fluid to prevent gas to be transported down-streams towards the patient P. Furthermore, the drip chamber 1 may comprise a membrane (320 in the embodiments of FIGS. 3 to 9) for filtering the medical fluid before it is delivered to the patient P.

FIG. 2 shows a view of a drip chamber 1 as it may be employed, for example, for the infusion, transfusion or parenteral nutrition. The drip chamber 1 comprises a first, top part 2 and a second, bottom part 3. The first part 2 comprises an inlet formed by a piercing needle 20 which may be inserted, in a piercing fashion, into a container 5, for example a flexible bag. The inlet 20, hence, provides a fluid connection to an up-stream container 5 on the inlet side of the drip chamber 1. The first part 2 further comprises a cap 21 to which the inlet 20 is connected.

The cap 21 of the first part is adjoined by a chamber element 30 of the second part 3. The chamber element 30 has a generally cylindrical, tubular shape and comprises a sufficient elasticity and flexibility such that the chamber element 30 may be squeezed in a squeezing direction S to perform a pumping action for causing fluid to flow into a chamber 300 formed by the chamber element 30 (see, for example, FIG. 3).

The first part 2 and the chamber element 30 of the second part 3 may for example be connected to each other by gluing them together. Herein, the first part 2 may be produced from a rigid plastic material which has an increased stiffness as compared to the stiffness of the chamber element 30. In particular, when squeezing the chamber element 30 in the transverse squeezing direction S, the first part 2 substantially is not deformed.

The chamber element 30, at its bottom end opposite the first part 2, is adjoined by a connector 31 which provides for a connection with a tube 4, for example a PVC tube. The connector 31 herein is integrally formed with the chamber element 30 by employing a two-component injection molding technique, as shall be subsequently described in more detail with regard to the embodiments of FIGS. 3 to 9.

In a first embodiment, shown in FIG. 3, a connector 31 substantially in the shape of a tube element is formed on the chamber element 30 by injection molding. The connector 31 is placed on a stub section 302 protruding from a bottom 301 of the chamber element 30. The connector 31 forms a reception opening 310 which diameter D2 matches the outer diameter of the tube 4 such that the tube 4 with an end 41 may be fitted into the reception opening 310 of the connector 31.

The stub section 302 at the bottom 301 of the chamber element 30, on its outer circumferential face, may have a cone-like shape. On its outer circumferential face, the stub section 302 is circumferentially surrounded by the connector 31 molded onto the stub section 302 such that the connector 31 is integrally connected to the stub section 302.

Within the stub section 302 an outlet opening 309 is formed providing a flow connection in-between the chamber 300 within the chamber element 30 and the tube 4 connected to the chamber element 30 via the connector 31. The outlet opening 309 has a diameter D1 which matches the diameter of the inner lumen 40 of the tube 4. Hence, when inserting the tube 4 into the reception opening 310 until it abuts the stub section 302, no step is formed in-between the outlet opening 309 of the stub section 302 and the inner lumen 40 of the tube 4, which reduces the risk for the occurrence of turbulences at the transition from the outlet opening 309 to the inner lumen 40 and hence decreases the risk for generating air bubbles in the down-stream flow.

The chamber element 30 is made of a first material, whereas the connector 31 is made of a second material different from the first material. In particular, the chamber element 30 is made of a material having a rather small modulus of elasticity, such that the chamber element 30 may be squeezed in the transverse squeezing direction S and for this is sufficiently elastic, flexible and resilient.

The connector 31, in contrast, comprises an increased stiffness as compared to the chamber element 30 in order to provide a reliable and durable connection to the tube 4. The material of the connector 31, in particular, has a higher modulus of elasticity than the material of the chamber element 30.

For example, the chamber element 30 may be fabricated from a polystyrene of a first type, whereas the connector 31 is made of a polystyrene of a second, harder type or a methyl methacrylate acrylonitrile butadiene styrene (MABS).

As shown in FIG. 3, at the bottom of the chamber 300 a membrane 320 is placed dividing the chamber 300 from the outlet opening 309. A medical fluid, hence, must pass through the membrane 320 to exit the chamber 300. In the embodiment of FIG. 3, the membrane 320 is held in-between ring-like fixing parts 321, 322 which receive a circumferential edge 323 of the membrane in-between them and, for example, may be glued to a bottom section 304 of the chamber element 30.

A second embodiment is shown in FIGS. 4 to 6.

Within this embodiment, the connector 31 comprises a stub section 314 forming a reception opening 310 for receiving the end 41 of the tube 4 therein. The stub section 314 is connected via a cone section 311 with a connecting section 312 and via the connecting section 312 to the chamber element 30. The cone section 311 tapers from the connecting section 312 towards the stub section 314 such that a fluid from the chamber 300 is guided towards the stub section 314 and the tube 4 arranged thereon.

The connecting section 312 circumferentially surrounds a bottom section 304 of the chamber element 30. The connecting section 312 herein in a ring-like fashion surrounds the bottom section 304, the bottom section 304 corresponding to a bottom edge region of the chamber element 30 at the lower end of the chamber 300.

Again, the connector 31 is integrally formed on the chamber element 30 by using injection molding. The chamber element 30 at the connector 31 may be formed from the materials stated previously.

In the embodiment of FIGS. 4 to 6, a membrane 320 may be arranged in-between an end face 305 of the bottom section 304 of the chamber element 30 and a face 313 at the connection location of the cone section 311 and the connecting section 312 of the connector 31. The end face 305 as well as the face 313 extend circumferentially on the chamber element 30 respectively the cone section 311 of the connector 31 and hold in-between them an edge 323 of the membrane 320.

The membrane 320 herein may be integrally fastened within the drip chamber 1 during injection molding of the connector 31 onto the chamber element 30 such that no separate fastening step for fastening the membrane 320 is necessary.

Another embodiment is shown FIGS. 7 to 9. This embodiment differs from the embodiment of FIGS. 4 to 6 in that a cone section 306 adjoins the bottom section 304 of the chamber element 30, the cone section 306 having a cone-like shape tapering towards a stub section 307 reaching into the stub section 314 of the connector 31.

At the stub section 307 of the chamber element 30, an outlet opening 309 is formed which has a diameter D1 corresponding to the diameter D1 of the inner lumen 40 of the tube 4 to be attached to the connector 31. The outlet opening 309 is adjoined by an insertion opening 308, which is in alignment with the reception opening 310 of the connector 31. In particular, the insertion opening 308 has a diameter D2 matching the diameter D2 of the reception opening 310 and the outer diameter D2 of the tube 4, such that the tube 4 with its end 41 can be inserted into the reception opening 310 and can be pushed forward until it reaches into the insertion opening 308 of the stub section 307 of the chamber element 30.

In a state in which the tube 4 is inserted into the connector 31, hence, the outlet opening 309 formed in the stub section 307 is in alignment with the inner lumen 40 of the tube 4 such that no step at the transition between the outlet opening 309 and the inner lumen 40 of the tube 4 is created, thus reducing the risk for turbulences and for generating air bubbles in the outlet flow flowing out of the drip chamber 1.

In addition, the reception opening 310 of the connector 31 and the insertion opening 308 on the chamber element 30 together form a tube receptacle which allows to glue the tube 4 to both the connector 31 and the chamber element 30 with its different materials. This will generate an additional tightness when the tube 4 gets glued, because the transition between the chamber element 30 and the connector 31 will be covered by the surface of the tube 4 and hence is sealed.

In the embodiment of FIGS. 7 to 9, the connector 31 overmolds the stub section 307, the cone section 306 and the bottom section 304 of the chamber element 30.

As shown in FIGS. 7, 8A and 9, a membrane 320 for filtering an outlet flow is fastened on a circumferential planar face 305′ formed on the inside of the cone section 306 of the chamber element 30. The face 305′ is arranged at a connection location in-between the cone section 306 and the bottom section 304. The membrane 320 may be fastened to the face 305′, for example by hot stamping, by another welding technique or by gluing.

Again, the chamber element 30 and the connector 31 may be made of different materials, examples of such materials given above.

The idea underlying the invention is not limited to the embodiments described above, but may be implemented also in entirely different embodiments.

In particular, the drip chamber with its chamber element and its connector may also have a different design than described above. Different integral connections in-between the chamber element and the connector are possible. For example, the connector not necessarily surrounds the chamber element at its bottom, but may also reach into the chamber element.

In addition, different materials than the ones described above are possible for forming the chamber element and the connector. For example, different thermoplastic materials may be used for producing the chamber element and the connector.

Alternative connection technologies for the chamber element 30 and connector 30 are possible by thermal welding, ultrasonic welding, solvent welding, and/or gluing.

### LIST OF REFERENCE NUMERALS

1 Drip chamber
2 First part
20 Inlet (piercing needle)
21 Cap
3 Second part
30 Chamber element
300 Chamber
301 Bottom
302 Neck portion
304 Bottom section
305 End face
305′ Face
306 Cone section
307 Stub section
308 Insertion opening
309 Outlet opening
31 Connector
310 Reception opening
311 Cone section
312 Connecting section
313 Face
314 Stub section
32 Membrane device
320 Membrane
321, 322 Fixing part
323 Membrane edge
4 Tube
40 Lumen
41 End
5 Container (flexible bag)
6 Stand
D1, D2 Inner diameter
F Flow direction
P Patient
S Squeezing direction

1. A drip chamber for administering a medical fluid, comprising:
a chamber element forming a chamber,
an inlet for letting a medical fluid into the chamber, and
a connector connected to the chamber element at an outlet of the drip chamber, wherein a tube is connectable to the connector for letting the medical fluid out of the chamber,
characterized in
that the connector is formed in one piece on the chamber element by using injection molding, wherein the chamber element is formed from a first material and the connector is formed from a second material which is different from the first material.

2. The drip chamber according to claim 1, characterized in that the first material has a smaller modulus of elasticity than the second material.

3. The drip chamber according to claim 1, characterized in that the first material and the second material are thermoplastic polymers having different moduli of elasticity.

4. The drip chamber according to claim 1, characterized in that the first material is a polystyrene or styrene butadiene block polymer of a first type and the second material is a polystyrene of a second type having a higher modulus of elasticity as compared to the polystyrene of the first type.

5. The drip chamber according to claim 1, characterized in that the first material is a polystyrene or styrene butadiene block polymer and the second material is a methyl methacrylate acrylonitrile butadiene styrene.

6. The drip chamber according to claim 1, characterized in that the connector is arranged at a side of the chamber element opposite the inlet.

7. The drip chamber according to claim 1, characterized in that the connector comprises a reception opening into which an end of the tube is insertable.

8. The drip chamber according to claim 7, characterized in that the reception opening adjoins an outlet opening formed in the connector or in the chamber element for providing a flow connection to the chamber, the reception opening having a larger diameter as compared to the diameter of the outlet opening.

9. The drip chamber according to claim 7, characterized in that the connector has a stub section forming the reception opening.

10. The drip chamber according to claim 9, characterized in that the stub section is connected to a bottom section of the chamber element via a cone section tapering towards the stub section.

11. The drip chamber according to claim 1, characterized in that the connector comprises a connecting section circumferentially surrounding the chamber element at its bottom section.

12. The drip chamber according to claim 1, characterized in that the chamber element comprises a stub section extending into the connector and forming an insertion opening for receiving an end of the tube, wherein the insertion opening adjoins an outlet opening formed in the stub section for providing a flow connection to the chamber, the insertion opening having a larger diameter as compared to the diameter of the outlet opening.

13. The drip chamber according to claim 1, characterized in that a membrane is arranged, with a circumferential membrane edge, in between a face of the chamber element and a face of the connector, the membrane separating the chamber from the outlet.

14. The drip chamber according to claim 1, characterized in that the membrane, with a circumferential membrane edge, is connected to a face of the chamber element or the connector, the membrane separating the chamber from the outlet.

15. A method for producing a drip chamber for administering a medical fluid, the drip chamber comprising:
a chamber element forming a chamber,
an inlet for letting a medical fluid into the chamber, and
a connector connected to the chamber element at an outlet, wherein a tube is connectable to the connector for letting the medical fluid out of the chamber,
characterized in
that the connector is formed in one piece with the chamber element using a two-component injection molding technique, wherein the chamber element is formed from a first material and the connector is formed from a second material which is different from the first material.
